(19) United States
(12) Patent Application Publication
(10) Pub. No.: US 2011/0104173 A1
(43) Pub. Date: May 5, 2011

(54) INHIBITION OF DEGRADATION OF EXTRACELLULAR MATRIX

(75) Inventors: Charmaine Simeonovic, Higgins (AU); Christopher Richard Parish, Campbell (AU); Andrew Ziolkowski, Cook (AU)

(73) Assignee: The Australian National University, Acton (AU)

(21) Appl. No.: 12/446,196

(22) PCT Filed: Oct. 22, 2007
(86) PCT No.: PCT/AU2007/001603

§ 371 Date: Jan. 10, 2011

(30) Foreign Application Priority Data
Oct. 20, 2006 (AU) 2006905854

(51) Int. Cl. A61K 31/702; A61K 39/395; A61K 39/00; A61P 37/00; A61P 37/06; A61P 3/10

(52) U.S. Cl. 424/141.1; 514/61; 424/184.1

(57) ABSTRACT

This application relates to a method of inhibiting the degradation of an extracellular matrix associated with islet beta cells, said method comprising contacting said extracellular matrix with an effective amount of a heparanase inhibitor.

### CROSS REFERENCE TO RELATED APPLICATION

This application claims the benefit of Australian Provisional Patent Application No. 2006905854 filed 20 Oct. 2006, which is incorporated herein by reference in its entirety.

### TECHNICAL FIELD

The present invention relates to the use of heparanase inhibitors in the treatment of conditions associated with extracellular matrix degradation such as insulitis or autoimmune Type-1 diabetes. In particular the invention relates to the improvement of transplantation outcomes for the treatment of insulitis or Type-1 diabetes.

### BACKGROUND OF THE INVENTION

Type 1 diabetes (T1D) is an autoimmune disease in which the insulin-producing beta cells of pancreatic islets are destroyed. In humans, this disease has an enormous impact on lifestyle and the imperfect control of hyperglycemia by exogenous insulin therapy inevitably leads to microvascular disease. This complication can ultimately result in kidney disease, heart disease, blindness and neuropathies leading to gangrene and the amputation of limbs. The clinical transplantation of pancreatic islets potentially offers an improved treatment for T1D because insulin can be delivered physiologically as the body requires it. In addition, this approach avoids the surgical complications associated with pancreas transplantation. Clinical islet transplantation, as a treatment for T1D, has progressed considerably in recent years with implementation of the Edmonton protocol. Despite this progress, the heavy use of immunosuppressive drugs required to prevent the rejection of the islet transplants has severely limited its application to only adult subjects whose diabetes has been difficult to control. Furthermore, in the long term, islet function is eventually lost and insulin therapy is again required. This graft failure is most likely due to toxicity of the immunosuppressive drugs used to prevent immunological rejection of the transplant and/or to recurrence of autoimmune disease. It is therefore essential that better anti-graft rejection/destruction strategies are developed to eliminate the need for toxic immunosuppressive drugs and thus preserve the health status of the subjects and the integrity of the transplant.

NOD (Non-obese diabetic) mice spontaneously develop diabetes due to autoimmune destruction of the insulin-producing beta cells present in the islets of the pancreas. The pathology of the autoimmune response initially involves the accumulation of non-invasive MNCs (mononuclear cells) such as T cells and macrophages around the periphery of the islets. This benign or non-destructive insulitis pathology switches to invasive insulitis (i.e. destructive autoimmunity), as female NOD mice grow older but the factors regulating this conversion are unclear (1). Morphometric studies have indicated that beta cell destruction occurs when >50% of islets in host pancreas have invasive insulitis and diabetes is seen when the insulin content of the pancreas reaches <10% of normal mice.

In clean NOD mouse colonies the incidence of diabetes in female mice can reach 80% and in male mice, 20% (1). The development of peri-islet insulitis and the subsequent onset of diabetes in NOD mice is a T cell-dependent process and adoptive transfer studies have demonstrated that both CD4⁺ and CD8⁺ T cells are required. This autoimmune T cell response is associated with a Th1-biased cytokine profile and is thought to be generated initially against a limited number of autoantigens but progresses through intra- and inter-molecular spreading to eventually involve multiple beta cell autoantigens i.e. proinsulin/insulin, GAD 65, IA-2 and Heat Shock Protein 65 (HSP65). Breakdown in immunoregulation resulting from an imbalance between populations of effector T cells and regulatory T cells has been identified as a major factor contributing to the onset of destructive autoimmunity.

The present invention relates to the discovery of the basement membrane surrounding islets in the pancreas acting as an immunological barrier during the benign or non-destructive insulitis phase, preventing intra-islet leukocyte invasion. In relation to this, the onset of destructive MNC infiltration correlates with local damage of the islet BM (basement membrane) perlecan (heparan sulfate proteoglycan) by activated MNC-derived heparanase. Further the present invention relates to the discovery that heparanase produced by alloreactive and autoreactive T cells plays a critical role in the immunological destruction of islet transplants due to rejection and disease recurrence, respectively. The inventors have further made the important discovery that the intra-islet extracellular matrix (ECM) is rich in heparan sulfate which can function in maintaining beta cell health. The progression of destructive insulitis and MNC infiltration within islets correlates with degradation of intra-islet heparan sulfate in the ECM. Therefore intra-islet heparan sulfate appears to be required for beta cell survival.

Consequently there is the need for treatments to combat the breakdown of the islet BM/ECM (basement membrane/extracellular matrix) heparan sulfate by heparanase. Furthermore there is the need for improved strategies for preventing transplant rejection in subjects suffering from Type-1 diabetes.

### SUMMARY OF THE INVENTION

According to a first aspect of the present invention, there is provided a method of inhibiting the degradation of extracellular matrix associated with islet beta cells, said method comprising contacting said extracellular matrix with an effective amount of a heparanase inhibitor.

In one embodiment the extracellular matrix may be selected from the group comprising the basement membrane, the peri-islet capsule, the intra-islet extracellular matrix or any combination thereof.

According to a second aspect of the present invention, there is provided a method of inhibiting the degradation of a heparan sulfate proteoglycan in extracellular matrix associated with islet beta cells, said method comprising contacting said extracellular matrix with an effective amount of a heparanase inhibitor.

In one embodiment, the heparan sulfate proteoglycan may be perlecan, type XVIII collagen or agrin.

According to a third aspect of the present invention, there is provided a method of treatment of an autoimmune condition in a subject, wherein said method comprises administering a therapeutically effective amount of a heparanase inhibitor to a subject.

In one embodiment, the condition may be selected from the group comprising insultits, Type-1 diabetes, rejection of pancreatic islet transplant or any combination thereof.

According to a fourth aspect of the present invention, there is provided a method of treatment of insulitis in a subject, wherein said method comprises administering a therapeutically effective amount of a heparanase inhibitor to a subject.

According to a fifth aspect of the present invention, there is provided a method treating or preventing the rejection of a transplant in a subject wherein said method comprises administering a therapeutically effective amount of a heparanase inhibitor to a subject.

In one embodiment the transplant is pancreatic islet transplantation.

According to a sixth aspect of the present invention, there is provided a method for reducing the level of immunosuppressive therapy associated with transplantation, wherein said method comprises administering a therapeutically effective amount of a heparanase inhibitor to a subject.

In one embodiment the transplantation is pancreatic islet transplantation.

According to a seventh aspect of the present invention, there is provided a method of treatment for diabetes in a subject wherein said method comprises administering a therapeutically effective amount of a heparanase inhibitor to a subject.

In one embodiment, the diabetes is recent-onset Type-1 diabetes.

According to an eighth aspect of the present invention, there is provided a process for the manufacture of a pharmaceutical composition comprising admixing a heparanase inhibitor with a pharmaceutically acceptable carrier.

According to a ninth aspect of the present invention, there is provided use of a heparanase inhibitor for the preparation of medicament for treatment of insulitis.

According to a tenth aspect of the present invention, there is provided use of a heparanase inhibitor for the preparation of medicament for treatment of diabetes.

In one embodiment the diabetes is recent-onset Type-1 diabetes.

According to an eleventh aspect of the present invention, there is provided use of a heparanase inhibitor for the preparation of medicament for treatment of transplant rejection.

In one embodiment the transplantation is pancreatic islet transplantation.

According to a twelfth aspect of the present invention, there is provided use of a heparanase inhibitor for the preparation of medicament for inhibiting the degradation of heparan sulfate in the islet extracellular matrix.

According to a thirteenth aspect of the present invention, there is provided use of a heparanase inhibitor for the preparation of medicament for inhibiting the degradation of heparan sulfate proteoglycan.

According to a fourteenth aspect of the present invention, there is provided use of a heparanase inhibitor for the preparation of medicament for inhibiting the rejection of a transplant in a subject.

According to a fifteenth aspect of the present invention, there is provided use of a heparanase inhibitor for the preparation of medicament for reducing the level of immunosuppressive therapy associated with transplantation.

In one embodiment of any one of the first, second or twelfth aspects the aspect, the extracellular matrix may be selected from the group comprising basement membrane, intra-islet extracellular matrix, peri-islet capsule or any combination thereof.

In one embodiment of any one of the fourth to the seventh aspects administration of the heparanase inhibitor may be systemic or regional. Administration may be pareneteral, intracavitary, intravesically, intramuscular, intraarterial, intravenous, subcutaneous, topical or oral.

The heparanase inhibitor may be administered in the form of a composition together with one or more pharmaceutically acceptable carriers, adjuvants or diluents.

According to a sixteenth aspect of the present invention there is provided a composition when used for the treatment or prevention of a condition associated with extracellular matrix degradation, wherein the composition comprises a heparanse inhibitor together with one or more pharmaceutically acceptable carriers, diluents or adjuvants.

According to a seventeenth aspect of the present invention there is provided a composition when used for the treatment or prevention of a condition associated with extracellular matrix degradation, wherein the composition comprises a heparanase inhibitor, together with at least one other immunosuppressant or anti-inflammatory agent and optionally with one or more pharmaceutically acceptable carriers, diluents or adjuvants.

The anti-inflammatory agent may be selected from the group comprising steroids, corticosteroids, COX-2 inhibitors, non-steroidal anti-inflammatory agents (NSAIDs), aspirin or any combination thereof.

In one embodiment the non-steroidal anti-inflammatory agent may be selected from the group comprising ibuprofen, naproxen, fenbufen, fenprofen, flurbiprofen, ketoprofen, dexketoprofen, tiaprofenic acid, azapropazone, diclofenac, aceclofenac, diflunasil, etodolac, indometacin, ketorolac, lornoxicam, mefanamic acid, meloxicam, nabumetone, phenylbutazone, piroxicam, rofecoxib, celecoxib, sulindac, tenoxicam, tolfenamic acid or any combination thereof.

The immunosuppressant agent may be selected from the group comprising alemtuzumab, azathioprine, ciclosporin, cyclophosphamide, lefunomide, methotrexate, mycophenolate mofetil, rituximab, sulfasalazine tacrolimus, sirolimus, or any combination thereof.

According to any one of the preceeding aspects, the heparanase inhibitor may be selected from the group comprising sulfated polysaccharides, phosphorothioate oligodeoxynucleotides, non-carbohydrate heparin mimetic polymers, sulfated malto-oligosaccharides, phosphosulfomannans, sulfated spaced oligosaccharides, sulfated linked cyclitols, sulfated oligomers of glycamino acids, pseudodisaccharides, siastatin B derivatives, uronic acid-type Gem-diamine 1-N-iminosugars, suramin and suramin analogues, fungal metabolites, diphenyl ether, carbazole, indole, benz-1,3-azole derivatives, 2,3-Dihydro-1,3-1H-isoindole-5-carboxylic acid derivatives, furanyl-1,3-thiazol-2-yl, benzoxazol-5-yl acetic acid, Poly(N-acryl amino acids), metabolites, derivatives or analogues thereof or any combination thereof. The heparanase inhibitor may be a monoclonal antibody. The heparanase inhibitor may be PI-88.

### BRIEF DESCRIPTION OF THE DRAWINGS

Preferred embodiments of the present invention will now be described, by way of example only, with reference to the accompanying drawings wherein:

FIG. 1: Alcian blue staining of heparan sulfate in the extracellular matrix of a (a) neonatal NOD/Lt islet and (b) adult prediabetic NOD/Lt islet. Note alcian blue staining of heparan sulfate in the islet basement membrane in (a).

FIG. 2: Immunofluorescence staining with (a) rabbit anti-mouse nidogen-1 and (b) rabbit anti-mouse perlecan shows the presence of (a) nidogen and (b) perlecan (a heparan sulfate proteoglycan or HSPG) in the basement membrane (see white indicator lines) of a NOD islet in the absence of destructive insulitis.

FIG. 3: PI-88 treatment prevents development of autoimmune diabetes in NOD/Lt mice. Treatment of prediabetic adult female NOD/Lt mice from 10.5 weeks of age, with the heparanase inhibitor PI-88 (10 mg/kg/day i.p.; 250 μg/0.2 ml/day in saline i.p.) (n=23) prevents the onset of clinical diabetes, compared to control mice treated with saline (0.2 ml/day i.p.) (n=25) and suggests that PI-88 prevents destructive insulitis. The incidence of diabetes in the holding female NOD/Lt mouse colony of the inventors is 60%.

FIG. 4: (a) Normal BALB/c islet showing a defined boundary due to a basement membrane (BM). * indicates the basement membrane. (b) In vivo administration of 10 μg of purified human platelet-derived heparanase/0.5 ml PBS via the pancreatic duct in normal BALB/c mice resulted in histological evidence of islets lacking a basement membrane (*) (in situ) at 24-48 hours post-delivery and indicates that normal islet morphology can therefore be disrupted by heparanase in vivo.

FIG. 5: (a) Immunohistochemical localisation of purified human platelet-derived heparanase (H) around the periphery of an islet and associated with pancreatic ducts in BALB/c pancreas after in vivo injection via the pancreatic duct; Rabbit anti-human heparanase polyclonal antibody. (b) Background staining in BALB/c pancreas from (a) in the absence of primary anti-heparanase antibody and in the presence of Phosphate Buffered Saline (diluent) and horseradish peroxidase (HRP)-conjugated goat anti-rabbit Ig.

FIG. 6: (a) Macroscopic appearance of control BALB/c islets after culture for 24 hours in 10% CO₂, 90% air. (b) Appearance of BALB/c islets after culture for 24 hours with human platelet-derived heparanase (20 μg/ml). Unlike control islets, heparanase treatment in vitro resulted in peripheral damage to the islets and in some cases, islet degradation (c).

FIG. 7: (a) Hematoxylin and eosin staining of prediabetic NOD/Lt female pancreas shows evidence of intact islets (i) as well as islets with non-destructive insulitis (ii) and destructive insulitis (iii). (b) Histological staining of the same pancreas specimen with Alcian blue/0.65M magnesium chloride/pH 5.8 detects heparan sulfate in an intact islet (i) and in the islet basement membrane and extracellular matrix(ii); during the progression of destructive insulitis into the islet cell mass, the HS component of the ECM becomes disrupted, as indicated by the broken blue staining in the remaining islet cell mass (iii).

FIG. 8: (a) PI-88 therapy prevents disease recurrence in islet isografts. Isografts of 450-500 female NODscid islets transplanted beneath the kidney capsule of diabetic NOD/Lt female mice return non-fasting blood glucose levels to the normal range (shaded region defines the normal range). Without further treatment in a control recipient (circles), hyperglycemia returned from day 3. In contrast, an NODscid islet isograft maintains normoglycemia for up to 14 days in a diabetic NOD mouse treated with PI-88 (10 mg/kg/day i.p.) from day 3 (squares). At the time of harvest the control isograft (b) showed aggressive autoimmune destruction (mononuclear cell infiltrate) and islet remnants (*) but the hematoxylin and eosin stained isograft from the PI-88-treated mouse (c) showed revascularised islets (\) with peri-islet accumulation of MNCs (*).

FIG. 9: Isolated islets dispersed into single cells are predominantly insulin-producing beta cells, as confirmed by immunofluorescence. In contrast to control islet cells that remained intact over a 2 day culture period, beta cells treated for 1 hr with bacterial heparitinases (heparinases) (I+II+III; 0.25 U/ml) died (a). Placement of treated cells on an ECM (produced in vitro by a cell line) was able to largely rescue the beta cells from heparitinase-induced cell death (a). These findings indicated that islet beta cells need cell-bound HS to survive. In support of this notion, bacterial heparinase-treated beta cells were efficiently rescued by providing cultures with 5-50 ug/ml heparin (*P<0.0001), a highly sulfated form of heparan sulfate (b).

FIG. 10: BALB/c (H-2^{d}) islet allograft from a PI-88 treated recipient CBA/H (H-2^{k}) mouse at 7 days post-transplant shows accumulation of mononuclear cells (*) around the periphery of islets (a), compared to a corresponding control islet allograft which shows more advanced islet destruction (\) at 7 days post-transplant (b). PI-88-treatment of the host therefore resulted in better preservation of the engrafted allogeneic islets.

### DEFINITIONS

Certain terms are used herein which shall have the meanings set forth as follows.

As used herein, the term “comprising” means “including principally, but not necessarily solely”. Furthermore, variations of the word “comprising”, such as “comprise” and “comprises”, have correspondingly varied meanings.

As used herein the terms “treating” and “treatment” refer to any and all uses which remedy a condition or symptoms, prevent the establishment of a condition or disease, or otherwise prevent, hinder, retard, or reverse the progression of a condition or disease or other undesirable symptoms in any way whatsoever.

As used herein the term “effective amount” includes within its meaning a non-toxic but sufficient amount of an agent or compound to provide the desired effect. The exact amount required will vary from subject to subject depending on factors such as the species being treated, the age and general condition of the subject, the severity of the condition being treated, the particular agent being administered and the mode of administration and so forth. Thus, it is not possible to specify an exact “effective amount”. However, for any given case, an appropriate “effective amount” may be determined by one of ordinary skill in the art using only routine experimentation.

As used herein the term “extracellular matrix associated with islet beta cells” refers to any extracellular components surrounding or substantially surrounding, but not necessarily in contact with, islet beta cells or islets per se. These components further comprise heparan sulfate and/or heparan sulfate proteoglycans, for example perlecan, type XVIII collagen or agrin. The term “extracellular matrix” includes within its meaning basement membranes.

As used herein, the term “alkyl” includes within its meaning monovalent (“alkyl”) and divalent (“alkylene”) straight chain or branched chain saturated aliphatic groups having from 1 to 10 carbon atoms. The alkyl group may be C₁₋₆ alkyl. The alkyl group may be C₁₋₄ alkyl. The alkyl group may be C₁₋₃ alkyl. Thus, for example, the term alkyl includes, but is not limited to, methyl, ethyl, 1-propyl, isopropyl, 1-butyl, 2-butyl, isobutyl, tert-butyl, amyl, 1,2-dimethylpropyl, 1,1-dimethylpropyl, pentyl, isopentyl, hexyl, 4-methylpentyl, 1-methylpentyl, 2-methylpentyl, 3-methylpentyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, 2-ethylpentyl, 3-ethylpentyl, heptyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,1,2-trimethylbutyl, 1,1,3-trimethylbutyl, 5-methylheptyl, 1-methylheptyl, octyl, nonyl, decyl, and the like.

The term “aryl” as used herein refers to monovalent (“aryl”) and divalent (“arylene”) single, polynuclear, conjugated and fused residues of aromatic hydrocarbons having from 6 to 14 carbon atoms. The aromatic group may be C₆₋₁₀ aromatic. Examples of aromatic groups include phenyl, naphthyl, phenanthrenyl, and the like. The aryl group may be optionally substituted, e.g., with one or more substituents independently selected from methyl, ethyl, halo, CF₃, CH₂OH, OH, O-methyl and O-ethyl.

### DETAILED DESCRIPTION OF THE PREFERRED EMBODIMENTS

It is to be understood at the outset, that the figures and examples provided herein are to exemplify, and not to limit the invention and its various embodiments.

In accordance with the present invention, compositions, methods and kits are provided for the inhibition of extracellular matrix degradation. The methods generally comprise the use of compositions comprising at least one heparanse inhibitor.

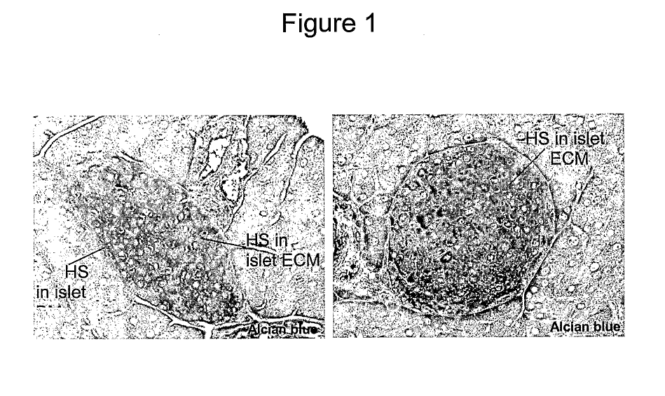

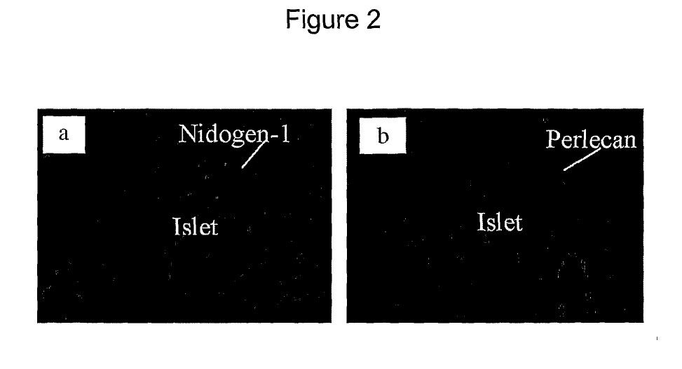

The extracellular matrix (ECM) is composed of a network of macromolecules which fills the extracellular space in tissue and provides molecular scaffolding for cells within different organs (3). ECMs are composed of structural proteins (e.g. collagen), specialized proteins (e.g. laminin) and proteoglycans (e.g. heparan sulfate proteoglycans including perlecan, type XVIII collagen or agrin (2)). In general, basement membranes (BMs) are thin sheets of extracellular matrix (ECM) which can surround groups of cells, thereby providing physical support and a major barrier to cell migration (3). Typically, they consist of protein and polysaccharide components. Heparan sulfate glycosaminoglycans represent the major polysaccharide component of BMs (4, 5). The inventors have identified that intra-islet ECM is enriched in heparan sulfate (see FIG. 1) and that the BM surrounding pancreatic islets contains perlecan (see FIG. 2(b)).

Accordingly, the inventors focused on perlecan as a key BM component and as an initial target for MNC-mediated degradation by heparanase. Perlecan is a heparan sulfate proteoglycan (HSPG) which consists of a core protein (400-470 kDa) with three attached molecules of the polysaccharide (glycosaminoglycan), heparan sulfate (HS) (6). HSPGs/perlecan interact with type IV collagen and laminin and thereby stabilise the overall BM structure. In the BM of blood vessels, perlecan largely contributes to the membrane's anionic charge (due to the negatively charged sulfate groups) and selective permeability (6).

The endoglycosidase (endo-beta-glucuronidase), heparanase (also known as heparinase), is the only known mammalian enzyme that can cleave the heparan sufate (HS) (also known as heparin sulfate) chains of HSPGs. Heparanase is produced as a proenzyme of approximately 65 kDa and requires proteolytic cleavage to two smaller polypeptides (8 kDa and 50 kDa) for formation of the active enzyme (7, 8). At least for T cells, heparanase expression appears to be regulated by proinflammatory cytokines and the enzyme can be ultimately bound to the cell surface by the mannose phosphate receptor (3). Heparanase has been found to play a vital role in the armoury needed by invading cells to degrade the ECM, particularly in metastasising tumours and tumour-associated angiogenesis (3).

### Degradation of Islet Heparan Sulfate by Heparanase and its Role in Beta Cell Destruction

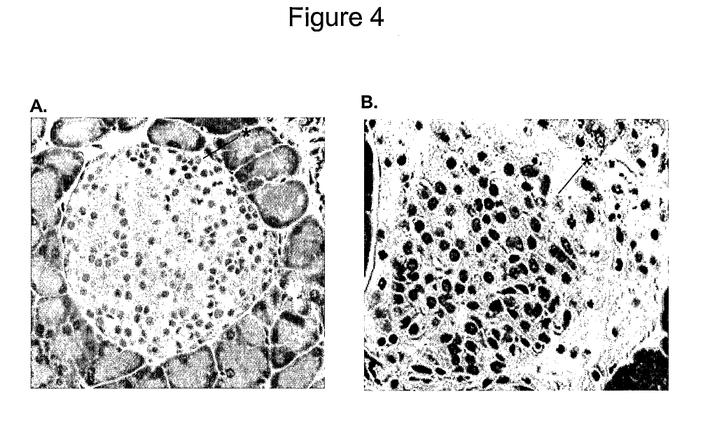

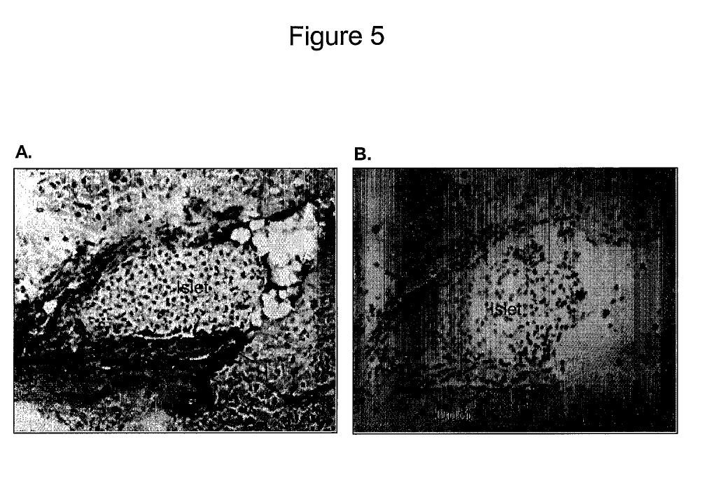

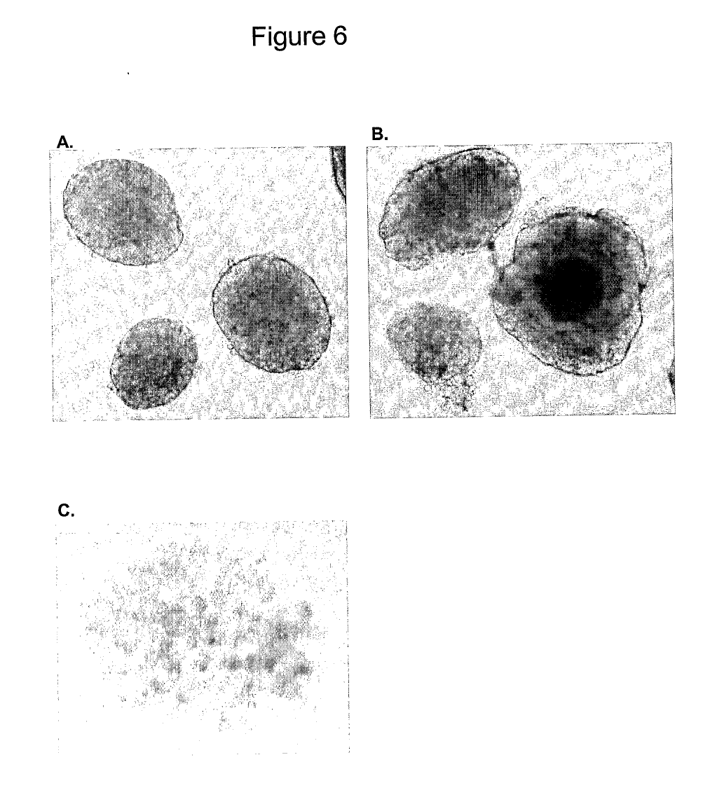

The inventors have found that the peri-islet capsule has properties consistent with that of a basal lamina or basement membrane (BM) which contains perlecan (heparan sulfate proteoglycan or HSPG). Furthermore, intra-islet infiltration was accompanied by major disruption of the basement membrane. Studies of tumor metastases have shown that tumor cell invasion occurs by breakdown of the underlying BM and/or extracellular matrix by degradative enzymes such as heparanase (3). Similarly the BM surrounding islets in the NOD/Lt pancreas acts as an immunological barrier during the non-destructive insulitis phase, preventing intra-islet leukocyte invasion. Onset of destructive MNC infiltration correlates with local damage of the islet basement membrane by activated MNC-derived heparanase. Once the basement membrane barrier is traversed, the inventors made the unique discovery that progression of the destructive insulitis correlates with disruption of heparan sulfate in the intra-islet extracellular matrix by heparanase and with beta cell demise. Heparanase-treatment of islets in vivo (FIGS. 4 and 5) and in vitro results in islet damage and in some cases complete islet destruction (FIG. 6). Beta cell survival is therefore dependent not only on an intact islet ECM-beta cell association but maintenance of intact BM and intra-islet ECM heparan sulfate.

In the case of islets transplanted beneath the kidney capsule, the graft becomes revascularised by a host-derived capillary network, originating from host blood vessels in the kidney parenchyma. The pathway taken by activated leukocytes during islet graft rejection and autoimmune destruction involves migration from newly formed intra-graft blood vessels or from nearby pre-existing renal blood vessels in the kidney tissue beneath the transplant. The recruitment of leukocytes to sites of inflammation requires activated T cells to traverse the vascular endothelium at nearby sites and move through the subendothelial BM into the adjacent tissue. Following leukocyte tethering to vascular endothelial cells and rolling of leukocytes, interaction with endothelial cell-bound chemokines, they traverse the vascular endothelium between the endothelial cells, and then move through the subendothelial BM by means of the degradative function of various enzymes such as MMPs and heparanase (9). Heparan sulfate acts as a vascular adhesion ligand, binder/immobiliser/transporter of chemokines and as a barrier to leukocyte migration in the subendothelial BM. Degradation of the BM heparan sulfate by heparanase is a critical and essential process for leukocyte migration. The activated T cells/leukocytes do not migrate from intra-islet capillaries but instead move from intragraft sites surrounding the islets, across the islet BM and into the islet cell mass. Such MNC migration requires heparanase-mediated degradation of BM HSPGs. Activated leukocytes, proinflammatory cytokine-stimulated endothelial cells and platelets produce heparanase (10, 11). Expression of heparanase activity has been found associated with extravasation of T cells across vascular BMs and development of inflammation in the central nervous system in rodents (12). Likewise lipopolysaccharride (LPS)-induced inflammation in rodents has been prevented by in vivo treatment with the heparanase inhibitior, PI-88. Intra-vital microscopy demonstrated leukocyte rolling and adherence to the vascular BM but no extravasation. These findings suggest that PI-88 could also inhibit the passage of activated MNCs into islet grafts.

### Susceptibility of Islet Transplants to Recurrence of Autoimmune Disease

Whereas the development of insulitis and diabetes onset in NOD mice is a T cell-dependent process requiring both CD4⁺ and CD8⁺ T cells, the recurrence of disease in NOD as well as NODscid islet isografts transplanted to diabetic NOD mice is mediated by CD4⁺ T cells and is prevented by depleting or non-depleting anti-CD4 mAb therapy. Since islet beta cells are class II Major Histocompatibility Complex (MHC)-ve, it appears that diabetes-associated beta cell-specific autoantigens are processed and presented in association with host MHC Class II by one or more intragraft antigen presenting cell (APC) populations, thereby leading to recognition by autoreactive CD4 T cells and indirect damage to islet beta cells. In contrast to anti-CD4 mAb therapy, co-stimulatory blockade with anti-CD154mAb protocols have either not prevented or only delayed disease recurrence in islet isografts. Although the induction of mixed hematopoietic chimerism in diabetic NOD mice treated with nonmyeloablative conditioning, protected NOD islet grafts from autoimmune damage, this experimental approach is unsuited for clinical islet transplantation. In general, prevention of disease recurrence in islet transplants is a formidable obstacle and remains a major concern for current clinical islet transplantation trials.

The invasion of autoreactive T cells into the transplant site, across the basement membrane of transplanted isogeneic islets and through the intra-islet ECM also is a heparanase-dependent processes.

Accordingly the present invention relates to the prevention of invasion of autoreactive T cells into the transplant site, across the basement membrane of transplanted isogeneic islets by a heparanase inhibitor such as PI-88.

### Allotransplantation of Islets

The rejection of pancreatic islet allografts results from the direct activation of anti-donor reactive T cells by donor-type passenger leukocytes passively carried in the transplant (13). The contribution of CD4⁺ and CD8⁺ T cells to the rejection process appears to be influenced by the donor/recipient strain combination, state of islet tissue differentiation and the presence/absence of class II MHC+ve duct epithelium within the islet transplant.

Studies have demonstrated prolonged survival of islet allografts, often accompanied by tolerance induction, following pretreatment of the islet tissue in vitro with high oxygen or short-term treatment of recipient mice with anti-CD8 mAb or anti-CD4+anti-CD8 mAbs, co-stimulatory blockade using murine CTLA4-Fc or anti-CD154 mAb with donor-specific transfusion or anti-CD154mAb combined with anti-ICOSmAb.

Other studies using immunosuppressive drugs alone or in combination with other agents, have demonstrated induction of stable islet allograft survival. In a number of these models, tolerance has been shown to depend on host regulatory CD4⁺CD25⁺ T cells, indicating an active process of immune regulation. However, in the situation of allogeneic hosts with autoimmune-induced diabetes, immunotherapies effective in preventing allograft rejection in conventional non-autoimmune mice have usually failed or at best, delayed rejection (14-16). This problem is due to islet allografts also being susceptible to autoimmune attack (17) and hence the need to target two independent mechanisms of destruction: rejection and recurrence of autoimmune disease.

These barriers have been overcome in NOD mice using heavy immunosuppressive protocols or donor-specific hematopoietic chimerism but such approaches are problematic (e.g. harmful side-effects) or unrealistic for clinical islet transplantation. For this reason, the heparanase-dependent mechanism of leukocyte migration across BMs and destruction of intra-islet heparan sulfate has been targeted for intervention therapy because it is a pathway common for both islet allograft rejection and recurrence of autoimmune disease in islet transplants. Indeed treatment of mice with heparins exhibiting some anti-heparanase activity has prolonged skin allograft survival in mice.

The present invention relates to surprising discovery that heparanase plays a role in islet allograft rejection. Furthermore the inventors have found that heparanase inhibitors such as PI-88 can delay the immune destruction of islet allografts in conventional mice and protect islet isografts in autoimmune diabetic NOD hosts. Thus, heparanase inhibitors may constitute a new therapeutic for clinical islet transplantation and may minimize or prevent the need for harmful immunosuppressive drugs.

### Heparanase Inhibitors

Heparanase is an endo-β-glucuronidase that cleaves the heparan sulfate side chains of proteoglycans that are found on cell surfaces and as a major component of the extracellular matrix and basement membrane surrounding cells. Several heparanase inhibitors have been isolated or synthesized including heparin and modified heparin derivatives, various natural and synthetic polyanionic polymers and smaller molecules presumed to act as transition state analogues. Various classes of molecules and specific examples thereof are discussed hereafter.

Heparanase inhibitors according to the present invention are selected from the group comprising sulfated polysaccharides, phosphorothioate oligodeoxynucleotides, non-carbohydrate heparin mimetic polymers, sulfated malto-oligosaccharides, phosphosulfomannans, sulfated spaced oligosaccharides, sulfated linked cyclitols, sulfated oligomers of glycamino acids, pseudodisaccharides, siastatin B derivatives, uronic acid-type Gem-diamine 1-N-iminosugars, suramin and suramin analogues, fungal metabolites, diphenyl ether, carbazole, indole and benz-1,3-azole derivatives. The heparanase inhibitor may also comprise a monoclonal antibody.

The sulfated polysaccharide is selected from the group comprising heparin, λ-carrageenan, κ-carrageenan, fucoidan, pentosan polysulfate, 6-O-carboxymethyl chitin III, laminarin sulfate, calcium spirulan and dextran sulfate.

Examples of non-carbohydrate heparin mimetic polymers are selected from compounds of Formula 1 to Formula 7 shown below.

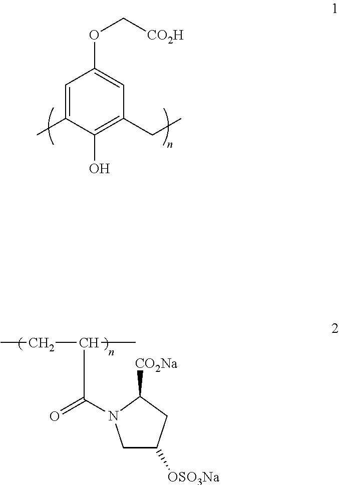

| 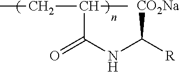 | |
|---|---|
| Formula: | R |
| 3 | H |
| 4 | iso-Bu |
| 5 | tert-Bu |
| 6 | (CH₂)₂CO₂Na |
| 7 | 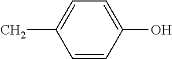 |

In formulae 1-7, n is less than or equal to 60.

Examples of sulfated malto-oligosaccharides are selected from the group comprising compounds of Formulae 8 to Formula 11 shown below.

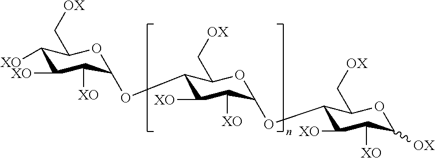

| Formula: | n |
|---|---|
| 8 | 2 |
| 9 | 5 |
| 10 | 0 |

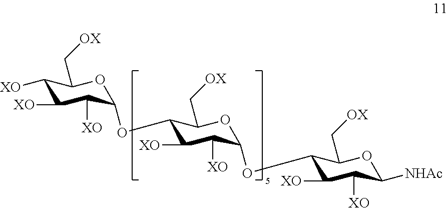

In formulae 8-11 X may be SO₃Na or H.

Examples of phosphosulfomannans are selected from the group comprising compounds of formulae 12 and 13, shown below. An example of such a compound is compound 13 (PI-88).

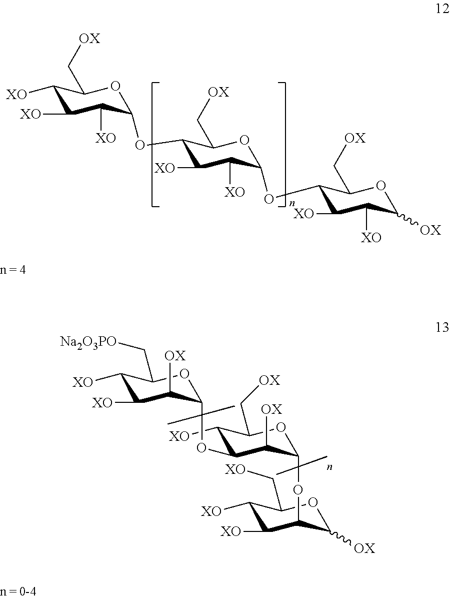

In formulae 12 and 13 X may be SO₃Na or H.

The invention includes analogues of PI-88 as shown below wherein R═SO₃Na or H. PI-88 is shown by Formula 13 above and analogues thereof are represented by Formulae 13a-13j below.

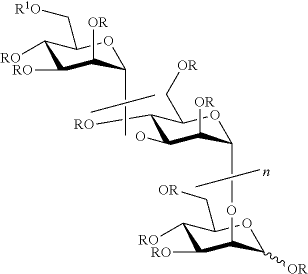

| Formula | n | R₁ |
|---|---|---|
| 13a | 0 | SO₃Na or H |
| 13b | 1 | SO₃Na or H |
| 13c | 2 | SO₃Na or H |
| 13d | 3 | SO₃Na or H |

In formulae 13a to 13d R may be SO₃Na or H

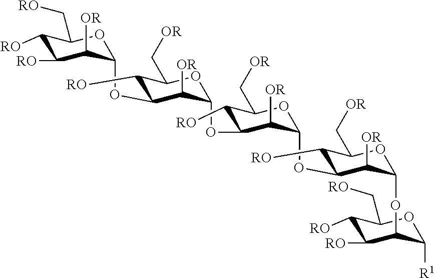

| Formula | R₁ |
|---|---|
| 13e | OBₙ |
| 13f | O(CH₂)₇CH₃ |
| 13g | OPEG₅₀₀₀—OMe |
| 13h | OPEG₂₀₀₀—OMe |
| 13i | NHCOCH₂OPh |
| 13j | NH—LC-Biotin |

An example of a sulfated “spaced” oligosaccharide is represented by general formula 16 as shown below

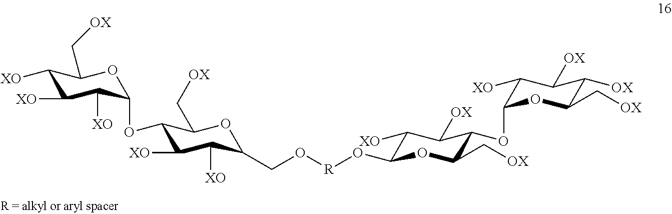

In formula 16 X may be SO₃Na or H. In addition, in formula 16 R may be a alkyl, aryl, alkylaryl, arylalkyl or an alkylarylaryl.

Other examples of a sulfated “spaced” oligosaccharide are represented by compounds of general formula 14 and formula 15 shown below.

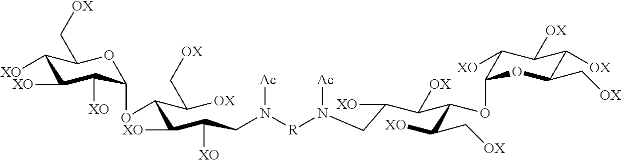

| Formula: | R |
|---|---|
| 14 | m-xylyl |
| 15 | (CH₂)₁₀ |

In formulae 14 and 15 X may be SO₃Na or H.

An example of a sulfated linked cyclitol may be selected from compounds represented by formulae 17, and 19. The compound represented by formula 18 is the starting reagent for making the cyclitol

| 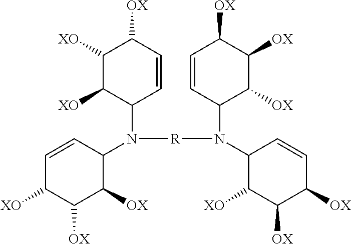 | |
|---|---|
| Formula | R |
| 17 | alkyl or aryl spacer |
| 19 | 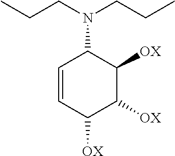 |

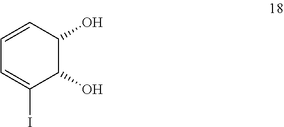

In formulae 17 and 19 X may be SO₃Na or H.

Examples of sulfated oligomers of glycamino acids are selected from the group comprising compounds of formulae 20, 21 and 22, shown below.

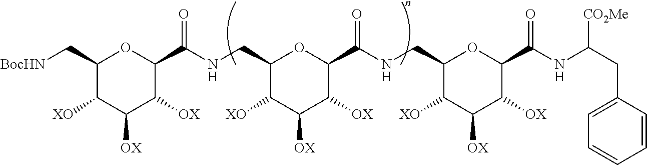

| Formula: | n |
|---|---|
| 20 | 2 |
| 21 | 1 |

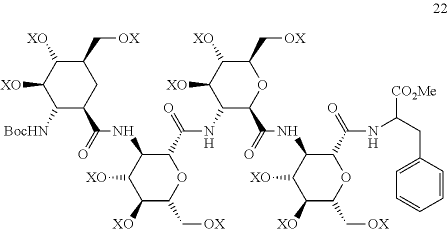

In formulae 20-22 X may be SO₃Na or H.

Examples of pseudodisaccharides may be selected from compounds of formulae 23 and 24, shown below, and salts thereof.

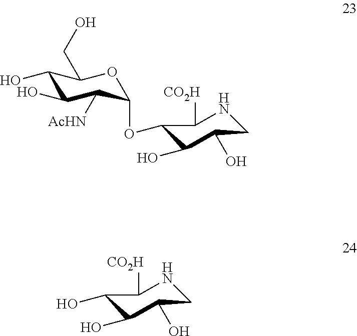

Examples of siastatin B derivatives may be selected from compounds of formulae 25, 26, 27 and 28, shown below.

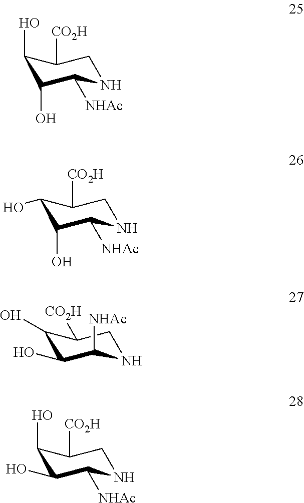

Examples of uronic acid-type Gem-diamine 1-N-iminosugars may be selected from compounds of formulae 29, 30 and 31, shown below, and salts thereof.

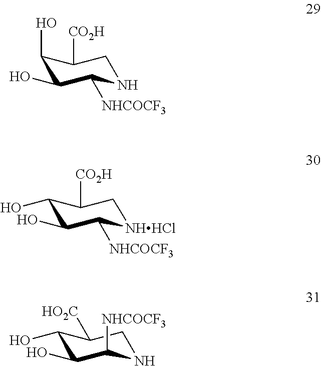

Examples of suramin and suramin analogues may be selected from compounds of formulae 32, 33, 34 and 35, shown below. Formulae 32 and 35 are alternate representations of the same compound.

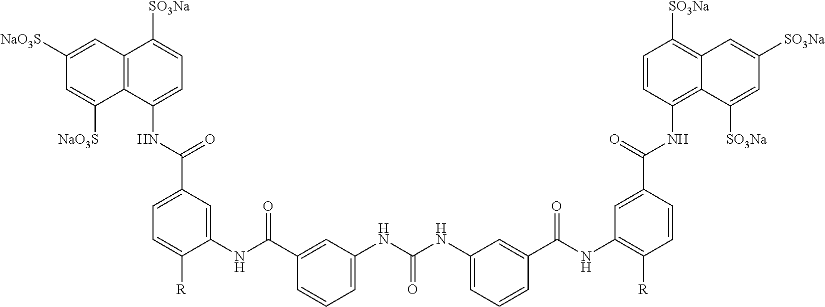

| Formula: | R |
|---|---|
| 32 | Me |
| 33 | Et |
| 34 | ^{t}Bu |

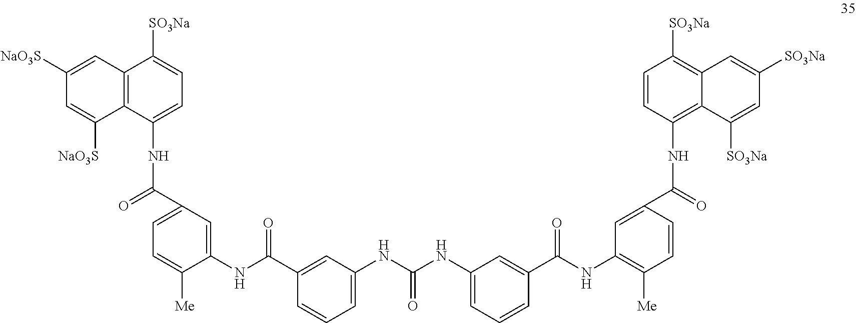

An example of a fungal metabolite may be selected from compounds of formulae 36, 37 and 38, shown below.

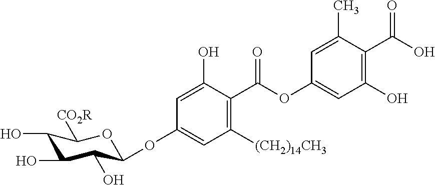

| Formula: | R |
|---|---|
| 36 | H |
| 37 | CH₃ |

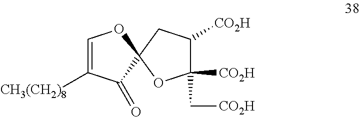

Examples of diphenyl ether, carbazole, indole and benz-1,3-azole derivatives may be selected from compounds of formulae 39, 39a, 40, 41 and 42, shown below, and salts thereof.

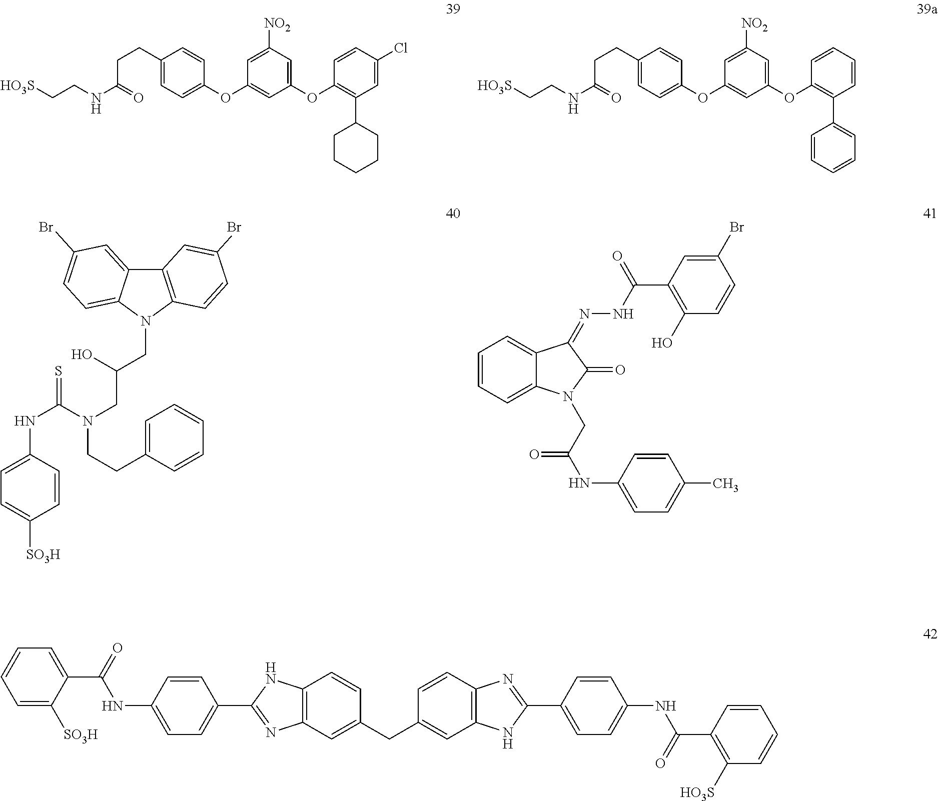

The heparanase inhibitor may also be selected from the following compounds.

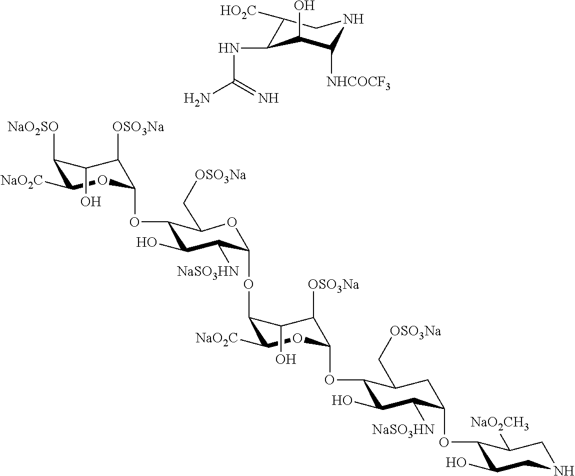

### Compositions and Methods of Treatment

Compounds for use in the present invention may be administered as compositions either therapeutically or preventively. In a therapeutic application, compositions are administered to a subject already suffering from a disease (e.g. early after disease onset), in an amount sufficient to cure or at least partially arrest the disease and its complications. The composition should provide a quantity of the compound or agent sufficient to effectively treat the subject.

In general, suitable compositions may be prepared according to methods which are known to those of ordinary skill in the art and accordingly may include a pharmaceutically acceptable carrier, diluent and/or adjuvant.

Methods for preparing administrable compositions are apparent to those skilled in the art, and are described in more detail in, for example, Remington's Pharmaceutical Science, 15th ed., Mack Publishing Company, Easton, Pa., hereby incorporated by reference herein.

Compositions for use in the present invention may include topical formulations and comprise an active ingredient together with one or more acceptable carriers, diluents, excipients and/or adjuvants, and optionally any other therapeutic ingredients. Formulations suitable for topical administration include liquid or semi-liquid preparations suitable for penetration through the skin to the site of where treatment is required, such as liniments, lotions, creams, ointments or pastes, and drops suitable for administration to the eye, ear or nose.

Drops for use in the present invention may comprise sterile aqueous or oily solutions or suspensions. These may be prepared by dissolving the active ingredient in an aqueous solution of a bactericidal and/or fungicidal agent and/or any other suitable preservative, and optionally including a surface active agent. The resulting solution may then be clarified by filtration, transferred to a suitable container and sterilised. Sterilisation may be achieved by: autoclaving or maintaining at 90° C.-100° C. for half an hour, or by filtration, followed by transfer to a container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Lotions for use in the present invention include those suitable for application to the skin or eye. An eye lotion may comprise a sterile aqueous solution optionally containing a bactericide and may be prepared by methods similar to those described above in relation to the preparation of drops. Lotions or liniments for application to the skin may also include an agent to hasten drying and to cool the skin, such as an alcohol or acetone, and/or a moisturiser such as glycerol, or oil such as castor oil or arachis oil.

Creams, ointments or pastes for use in the present invention are semi-solid formulations of the active ingredient for external application. They may be made by mixing the active ingredient in finely-divided or powdered form, alone or in solution or suspension in an aqueous or non-aqueous fluid, with a greasy or non-greasy basis. The basis may comprise hydrocarbons such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol such as propylene glycol or macrogols.

The composition may incorporate any suitable surfactant such as an anionic, cationic or non-ionic surfactant such as sorbitan esters or polyoxyethylene derivatives thereof. Suspending agents such as natural gums, cellulose derivatives or inorganic materials such as silicaceous silicas, and other ingredients such as lanolin, may also be included.

The compositions may also be administered in the form of liposomes. Liposomes are generally derived from phospholipids or other lipid substances, and are formed by mono- or multi-lamellar hydrated liquid crystals that are dispersed in an aqueous medium. Any non-toxic, physiologically acceptable and metabolisable lipid capable of forming liposomes can be used. The compositions in liposome form may contain stabilisers, preservatives, excipients and the like. The preferred lipids are the phospholipids and the phosphatidyl cholines (lecithins), both natural and synthetic. Methods to form liposomes are known in the art, and in relation to this is specific reference is made to: Prescott, Ed., Methods in Cell Biology, Volume XIV, Academic Press, New York, N.Y. (1976), p. 33 et seq., the contents of which are incorporated herein by reference.

The compositions may also be administered in an aerosol form (such as liquid or powder) suitable for administration by inhalation, such as by intranasal inhalation or oral inhalation.

### Combination Regimens

Therapeutic advantages may be realised through combination regimens. Those skilled in the art will appreciate that the heparanse inhibitors disclosed herein may be administered as part of a combination therapy approach to the treatment of insulitis and/or Type 1 diabetes. In combination therapy the respective agents may be administered simultaneously, or sequentially in any order. When administered sequentially, it may be preferred that the components be administered by the same route.

Alternatively, the components may be formulated together in a single dosage unit as a combination product. Suitable agents which may be used in combination with the compositions of the present invention will be known to those of ordinary skill in the art.

Methods of treatment according to the present invention may be applied in conjunction with conventional therapy. Conventiopnal therapy may comprise treatment of islets before transplantation (e.g. with high oxygen). Conventional therapy may also comprise anti-inflammatory therapy, immonunosupression therapy, surgery, or other forms of medical intervention.

Examples of anti-inflammatory agents include steroids, corticosteroids, COX-2 inhibitors, non-steroidal anti-inflammatory agents (NSAIDs), aspirin or any combination thereof. The non-steroidal anti-inflammatory agent may be selected from the group comprising ibuprofen, naproxen, fenbufen, fenprofen, flurbiprofen, ketoprofen, dexketoprofen, tiaprofenic acid, azapropazone, diclofenac, aceclofenac, diflunasil, etodolac, indometacin, ketorolac, lornoxicam, mefanamic acid, meloxicam, nabumetone, phenylbutazone, piroxicam, rofecoxib, celecoxib, sulindac, tenoxicam, tolfenamic acid or any combination thereof.

Examples of immunosuppressive agents include alemtuzumab, azathioprine, ciclosporin, cyclophosphamide, lefunomide, methotrexate, mycophenolate mofetil, rituximab, sulfasalazine tacrolimus, sirolimus, or any combination thereof.

Compounds and compositions disclosed herein may be administered either therapeutically or preventively. In a therapeutic application, compounds and compositions are administered to a patient already suffering from a condition, in an amount sufficient to cure or at least partially arrest the condition and its symptoms and/or complications. The compound or composition should provide a quantity of the active compound sufficient to effectively treat the patient.

### Dosages

The therapeutically effective dose level for any particular subject will depend upon a variety of factors including: the disorder being treated and the severity of the disorder; activity of the compound or agent employed; the composition employed; the age, body weight, general health, sex and diet of the subject; the time of administration; the route of administration; the rate of sequestration of the agent or compound; the duration of the treatment; drugs used in combination or coincidental with the treatment, together with other related factors well known in medicine.

One skilled in the art would be able, by routine experimentation, to determine an effective, non-toxic amount of agent or compound which would be required to treat applicable diseases. Generally, an effective dosage is expected to be in the range of about 0.01 mg to about 100 mg per kg body weight per 24 hours; typically, about 0.02 mg to about 90 mg per kg body weight per 24 hours; about 0.03 mg to about 80 mg per kg body weight per 24 hours; about 0.04 mg to about 70 mg per kg body weight per 24 hours; about 0.05 mg to about 60 mg per kg body weight per 24 hours; about 0.06 mg to about 50 mg per kg body weight per 24 hours. More typically, an effective dose range is expected to be in the range about 0.07 mg to about 40 mg per kg body weight per 24 hours; about 0.08 mg to about 30 mg per kg body weight per 24 hours; about 0.09 mg to about 25 mg per kg body weight per 24 hours; about 0.1 mg to about 20 mg per kg body weight per 24 hours.

Alternatively, an effective dosage may be up to about 500 mg/m². Generally, an effective dosage is expected to be in the range of about 25 to about 500 mg/m², preferably about 25 to about 350 mg/m², more preferably about 25 to about 300 mg/m², still more preferably about 25 to about 250 mg/m², even more preferably about 50 to about 250 mg/m², and still even more preferably about 75 to about 150 mg/m².

Typically, in therapeutic applications, the treatment would be for the duration of the disease state.

Further, it will be apparent to one of ordinary skill in the art that the optimal quantity and spacing of individual dosages will be determined by the nature and extent of the disease state being treated, the form, route and site of administration, and the nature of the particular individual being treated. Also, such optimum conditions can be determined by conventional techniques.

It will also be apparent to one of ordinary skill in the art that the optimal course of treatment, such as, the number of doses of the composition given per day for a defined number of days, can be ascertained by those skilled in the art using conventional course of treatment determination tests.

### Routes of Administration

The compositions of the invention may be in a form suitable for administration by injection, in the form of a formulation suitable for oral ingestion (such as capsules, tablets, caplets, elixirs, for example), in the form of an ointment, cream or lotion suitable for topical administration, in a form suitable for delivery as an eye drop, in an aerosol form (such as liquid or powder) suitable for administration by inhalation via the lung, such as by intranasal inhalation or oral inhalation, in a form suitable for parenteral (e.g., intravenous, intraspinal, subcutaneous or intramuscular), administration, that is, subcutaneous, intramuscular or intravenous injection.

### Carriers, Diluents, Excipients and Adjuvants

Carriers, diluents, excipients and adjuvants must be “acceptable” in terms of being compatible with the other ingredients of the composition, and not deleterious to the recipient thereof. Such carriers, diluents, excipient and adjuvants may be used for enhancing the integrity and half-life of the compositions of the present invention. These may also be used to enhance or protect the biological activities of the compositions of the present invention.

Examples of pharmaceutically acceptable carriers or diluents are demineralised or distilled water; saline solution; vegetable based oils such as peanut oil, safflower oil, olive oil, cottonseed oil, maize oil, sesame oils, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrolidone; agar; gum tragacanth or gum acacia, and petroleum jelly. Typically, the carrier or carriers will form from 10% to 99.9% by weight of the compositions.

The carriers may also include fusion proteins or chemical compounds that are covalently bonded to the compounds of the present invention. Such biological and chemical carriers may be used to enhance the delivery of the compounds to the targets or enhance therapeutic activities of the compounds. Methods for the production of fusion proteins are known in the art and described, for example, in Ausubel et al (In: Current Protocols in Molecular Biology. Wiley Interscience, ISBN 047 150338, 1987) and Sambrook et al (In: Molecular Cloning: A Laboratory Manual, Cold Spring Harbor Laboratories, New York, Third Edition 2001).

The compositions of the invention may be in a form suitable for administration by injection, in the form of a formulation suitable for oral ingestion (such as capsules, tablets, caplets, elixirs, for example), in the form of an ointment, cream or lotion suitable for topical administration, in a form suitable for delivery as an eye drop, in an aerosol form suitable for administration by inhalation, such as by intranasal inhalation or oral inhalation, in a form suitable for parenteral administration, that is, subcutaneous, intramuscular or intravenous injection.

For administration as an injectable solution or suspension, non-toxic parenterally acceptable diluents or carriers can include, Ringer's solution, isotonic saline, phosphate buffered saline, ethanol and 1,2 propylene glycol.

Some examples of suitable carriers, diluents, excipients and/or adjuvants for oral use include peanut oil, liquid paraffin, sodium carboxymethylcellulose, methylcellulose, sodium alginate, gum acacia, gum tragacanth, dextrose, sucrose, sorbitol, mannitol, gelatine and lecithin. In addition these oral formulations may contain suitable flavouring and colourings agents. When used in capsule form the capsules may be coated with compounds such as glyceryl monostearate or glyceryl distearate which delay disintegration.

Solid forms for oral administration may contain binders acceptable in human and veterinary pharmaceutical practice, sweeteners, disintegrating agents, diluents, flavourings, coating agents, preservatives, lubricants and/or time delay agents. Suitable binders include gum acacia, gelatine, corn starch, gum tragacanth, sodium alginate, carboxymethylcellulose or polyethylene glycol. Suitable sweeteners include sucrose, lactose, glucose, aspartame or saccharine. Suitable disintegrating agents include corn starch, methylcellulose, polyvinylpyrrolidone, guar gum, xanthan gum, bentonite, alginic acid or agar. Suitable diluents include lactose, sorbitol, mannitol, dextrose, kaolin, cellulose, calcium carbonate, calcium silicate or dicalcium phosphate. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

Liquid forms for oral administration may contain, in addition to the above agents, a liquid carrier. Suitable liquid carriers include water, oils such as olive oil, peanut oil, sesame oil, sunflower oil, safflower oil, arachis oil, coconut oil, liquid paraffin, ethylene glycol, propylene glycol, polyethylene glycol, ethanol, propanol, isopropanol, glycerol, fatty alcohols, triglycerides or mixtures thereof.

Suspensions for oral administration may further comprise dispersing agents and/or suspending agents. Suitable suspending agents include sodium carboxymethylcellulose, methylcellulose, hydroxypropylmethyl-cellulose, poly-vinyl-pyrrolidone, sodium alginate or acetyl alcohol. Suitable dispersing agents include lecithin, polyoxyethylene esters of fatty acids such as stearic acid, polyoxyethylene sorbitol mono- or di-oleate, -stearate or -laurate, polyoxyethylene sorbitan mono- or di-oleate, -stearate or -laurate and the like. The emulsions for oral administration may further comprise one or more emulsifying agents. Suitable emulsifying agents include dispersing agents as exemplified above or natural gums such as guar gum, gum acacia or gum tragacanth.

### Timing of Therapies

Those skilled in the art will appreciate that the compositions may be administered as a single agent or as part of a combination therapy approach to the treatment of autoimmune diseases, such as insulitis and/or Type 1 diabetes at diagnosis or subsequently thereafter, for example, as follow-up treatment or consolidation therapy as a complement to currently available therapies for such diseases. The compositions may also be used as preventative therapies for subjects who are genetically or environmentally predisposed to developing such diseases.

The present invention will now be further described in greater detail by reference to the following specific examples, which should not be construed as in any way limiting the scope of the invention.

### EXAMPLES

### Example 1

### Role of Heparanase in the Initiation of Destructive Insulitis and Clinical Diabetes

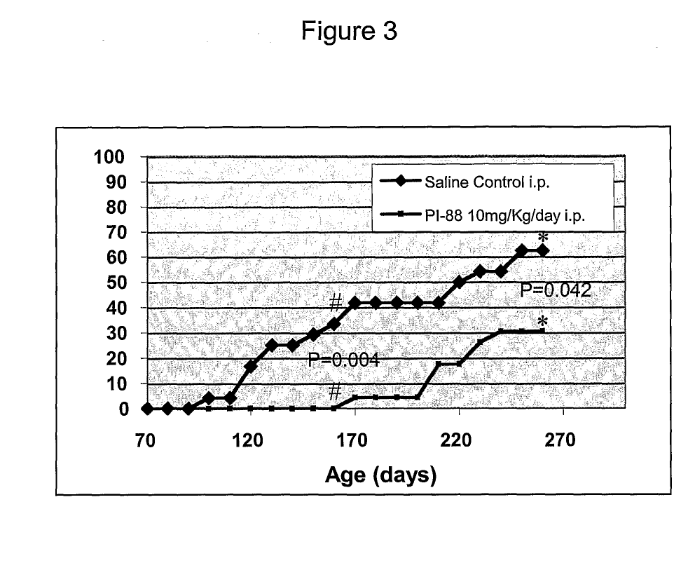

Studies conducted by the inventors have shown that heparanase transcripts are increased 7-fold in prediabetic and diabetes-onset NOD mice, compared to neonatal NOD mice; only background levels were detected in normal CBA/H mice (see TABLE 1). These results have been strengthened by the demonstration that treatment of NOD/Lt female mice from 10-11 weeks of age with the heparanase inhibitor PI-88 (9) prevents the onset of clinical diabetes in mice up to 24 weeks of age (see FIG. 3). Furthermore, the inventors have found that delivery of purified human platelet-derived heparanase to conventional (non-autoimmune) mice via the pancreatic duct in vivo, can result in disruption of islet BM and intra-islet HS in situ (see FIG. 4). These findings are consistent with a role for heparanase in the initiation of destructive insulitis in NOD mice and demonstrates the capacity of PI-88 to protect NOD mice from onset of clinical diabetes.

TABLE 1
Real-time RT-PCR analysis shows 7-fold upregulation of heparanase
mRNAs in islets from female prediabetic NOD/Lt mice and from
female NOD/Lt mice at diabetes-onset compared to islets isolated
from female NOD/Lt neonates (reference sample)

| | Relative Heparanase |
|---|---|
| Islet sample | mRNA expression* |
| 4-5 wk neonatal NOD/Lt | 1.0 |
| Prediabetic NOD/Lt | 7.06 ± 2.61 |
| Onset-diabetic NOD/Lt | 6.85 ± 0.58 |
| CBA/H (conventional mouse strain) | 1.24 ± 0.78 |

Islets from conventional CBA/H mice were used as a negative control. The data shows the mean ± SE for 3 individual series of samples.

### Example 2

Kinetics of Intragraft Expression of Heparanase mRNA in Islet Isografts Undergoing Autoimmune Destruction in Diabetic NOD Mice

Conventionally, the histopathology of pre-diabetic NOD female pancreas and of the autoimmune destruction of isogeneic islets transplanted to diabetic NOD mice shows that the entry of autoreactive T cells and other MNCs into the islets does not occur via the intra-islet vasculature. Instead, the invading leukocytes move from peri-islet locations into the islet cell mass after breakdown of the islet BM by degradative enzymes such as heparanase, produced locally by the activated leukocytes at the islet BM interface. Thereafter progression of the MNC infiltrate results in degradation of intra-islet ECM heparan sulfate and islet destruction.

The investigation of whether this enzyme-dependent mechanism of leukocyte invasion plays a role in the autoimmune destruction of islet isografts in diabetic NOD mice comprises typical experiments such as examination of the intragraft expression of heparanase transcripts in islet isografts harvested at various times post-transplant. The inventors have isolated donor islets from the pancreas of NODscid female mice at 6-8 weeks of age by collagenase digestion, using the intraductal collagenase infusion method (4-5 donor mice/islet isolation). Freshly isolated NODscid islets were transplanted beneath the kidney capsule of diabetic NOD/Lt female mice (250 islets/graft) in recipient NOD/Lt strain blood clots, 50-100 islets/clot (22). By using immunoincompetent NODscid donor mice for preparing the islets, instead of young NOD/Lt female mice, the possibility of passively carrying over donor insulitis-derived MNCs or islets already damaged by donor insulitis, to the transplant site was eliminated. Hence only a host-derived immune response is generated at the graft site for subsequent analysis. Isografts of fetal NODscid skin (derived from 17 day gestation fetal donors) or NODscid adult thyroid transplanted beneath the kidney capsule served as intact control grafts (not susceptible to autoimmune damage) and isolated NODscid islets and normal NOD/Lt kidney tissue served as additional negative controls.

Grafts were harvested at 3, 4, 5, 6, 8, 10 and 14 days post-transplant; the majority of each graft was frozen in liquid nitrogen for subsequent RNA extraction and the remainder was frozen in liquid freon for immunohistochemistry or fixed in 10% neutral-buffered formalin for histology. RNA was extracted using the guanidine isothiocyanate/caesium chloride method. All samples for comparison were reverse transcribed using the same reaction mix with oligo dT priming. Real-time RT-PCR was performed using validated primers/probe sets (Applied Biosystems). The expression of heparanase mRNA in tissue samples was analysed quantitatively.

The real-time RT-PCR method established in the inventors' lab uses a Taqman fluorogenic probe (6-FAM for target gene and endogenous reference gene (ubiquitin-conjugating enzyme E2D1 (UBC)) for PCR product measurement. The relative amount of target gene transcripts is calculated according to standard procedures (using C_{T} values for test genes and UBC). The efficiency of amplification for each primer/probe set is firstly optimized by testing a limited range of primer/probe concentrations with a standard amount of input cDNA. Linear regression analysis incorporated in the LinRegPCR programme is then used to calculate the PCR efficiency and correlation coefficient for the line of best fit for amplification plots; this information is used to identify the optimal primer/probe concentration. Using these conditions the housekeeping gene (UBC) and target gene are amplified with the same efficiency in test cDNAs. This permits compensation for different amounts in input cDNA and relative quantitation of test PCR product between samples using the comparative C_{T} method.

The preliminary studies of the inventors indicate that heparanase mRNA expression is upregulated approximately 4-fold during peak expression (at 5-6 days pos-transplant) during the autoimmune destruction of NODscid islet isografts (see TABLE 2 below).

TABLE 2
Real-time RT-PCR analysis shows upregulation in heparanase
mRNAs in NODscid islet isografts undergoing autoimmune
destruction in diabetic NOD/Lt mice*

| | Relative Heparanase mRNA expression | | | | | |
|---|---|---|---|---|---|---|
| | at time post-transplant (days)** | | | | | |
| Isograft to diabetic NOD | d 3 | d 4 | d 5 | d 6 | d 10 | d 14 |
| NODscid islets | 1.54 | 2.47 | 4.53 | 4.54 | 2.16 | 1.89 |
| Fetal NODscid skin* | 1.00 | 0.96 | 0.85 | 1.01 | 0.38 | 0.42 |

*Fetal NODscid skin isografts (NOT susceptible to autoimmune disease) were transplanted beneath the kidney capsule of diabetic NOD/Lt female mice as intact background control isografts.
**These data are representative of two independent series of isograft samples.

### Example 3

### Expression of Heparan Sulfate (HS) in NOD Islets In Situ and Degradation of Intra-Islet HS During Progression of Destructive Insulitis

In addition to the presence of perlecan/HS in the islet BM, HSPGs are also components of the more widely distributed ECM. ECMs consist of a network of macromolecules that function by filling the extracellular space in tissues and by providing a scaffolding for cells of a particular tissue and on which invading leukocytes can migrate (3). Indeed beta cell survival and function has been shown to depend on preservation of their interaction with the intra-islet ECM (23,24). It is therefore possible that heparanase not only facilitates the entry of activated MNCs across the islet BM but also degrades the intra-islet ECM, thereby reducing the viability of nearby beta cells as well as facilitating the migration of invading MNCs.

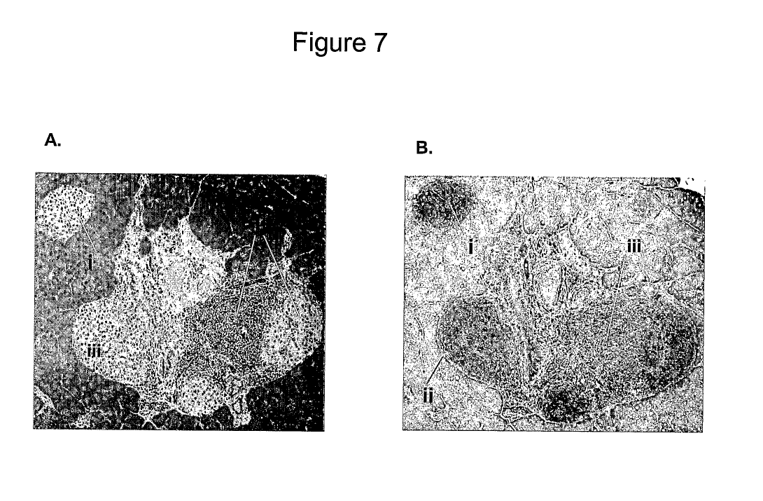

A typical experiment to confirm the relationship of islet-associated heparan sulfate and heparanase to islet integrity comprises harvesting pancreas from neonatal, prediabetic (±PI-88 treatment), diabetes-onset and diabetic (2-4 weeks post-onset) NOD/Lt mice as well as harvesting NODscid islets and Nodscid islet isografts from diabetic NOD mice (±PI-88 treatment) for fixation in 10% neutral-buffered formalin. Heparan sulfate (HS) is localised in formalin-fixed sections by histological staining with alcian blue/0.65M magnesium chloride/pH 5.8 (conditions which define HS specificity) (25) (see FIG. 7., page 19). This analysis has ascertained that HS is restricted to the islet BM, is distributed in the intra-islet ECM and is damaged during autoimmune injury.

### Example 4

### Islet Damage Induced by Exogenous Heparanase and Effect of PI-88 Therapy

Heparanase can be purified from human platelets (26, 27); platelet-derived heparanase has been shown to rapidly cleave heparan sulfate (HS) from endothelial cells and this activity is pH-dependent (26). Studies conducted by the inventors have shown that in vivo delivery of purified human platelet heparanase via the pancreatic duct of BALB/c mice can result in loss of normal islet morphology (see FIG. 4 and FIG. 5). A typical experiment ascertaining whether heparanase alone can induce damage to BALB/c islets or NODscid islets comprises the incubation of isolated islets overnight with purified human platelet-derived heparanase (10-20 μg/ml; see FIG. 6). Control islets were treated with phosphate-buffered saline (PBS). Thereafter the islets were examined microscopically/histologically to show heparanase-induced islet damage/destruction.

### Example 5

Expression of Heparanase mRNA and Protein during Islet Allograft Rejection

Since the studies conducted by the inventors suggest that heparanase plays an important role in the autoimmune damage of islets in situ and after transplantation, it was necessary to investigate whether heparanase functions in leukocyte migration/recruitment to the graft site and intra-islet invasion during the rejection of islet allografts. In the situation where islet allografts are implanted beneath the kidney capsule, heparanase can play an essential role in the (i) infiltration of alloreactive T cells across the islet BM into the islet cell mass (ii) extravasation of activated leukocytes from renal blood vessels and possibly from some host-derived intra-graft vasculature and (iii) destruction of intra-islet heparan sulfate. A typical experiment comprises analysis of the intragraft expression of heparanase transcripts from BALB/c (H-2^{d}) islet allografts harvested at 3-14 days post-trans-plant to CBA/H (H-2^{k}) recipient mice (see TABLE 3). Heparanase mRNA expression was upregulated approximately 3- to 4-fold during peak expression (at 5-7 days post-transplant) during islet allograft rejection (see TABLE 3 below).

TABLE 3
Real-time RT-PCR analysis shows 2-8-fold upregulation
of heparanase mRNAs in BALB/c (H-2^{d}) islet allografts undergoing
rejection in CBA/H (H-2^{k}) mice

| | Relative Heparanase mRNA expression | | | | | |
|---|---|---|---|---|---|---|
| | at time post-transplant (days) | | | | | |
| Transplant to CBA/H mice | d 3 | d 4 | d 5 | d 6 | d 7 | d 8 |
| BALB/c islet allograft | 1.49 | 2.11 | 3.27 | 2.55 | 4.08 | 2.52 |
| CBA/H islet isograft* | 1.00 | 1.53 | 1.14 | 1.07 | 0.47 | 0.92 |

Intact CBA/H islet isografts served as background controls.

### Example 6

Effect of Heparanase Inhibition with PI-88 on Islet Isograft Survival/Function in Diabetic NOD Mice

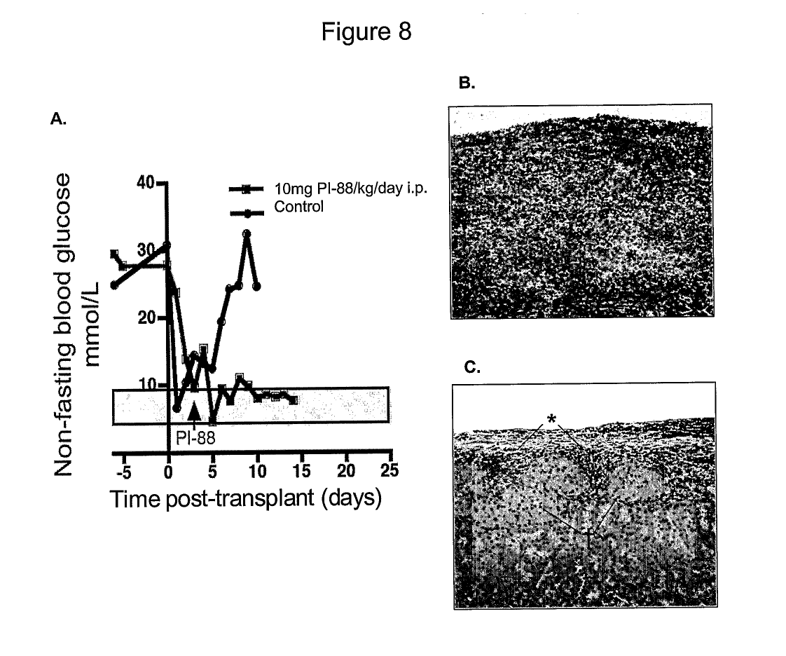

The finding that PI-88 treatment of prediabetic NOD mice prevents the onset of clinical diabetes (see FIG. 3) and hence destructive insulitis, strongly indicates that inhibition of heparanase activity is immunoprotective for islets in situ. Consequently in vivo treatment of transplanted mice with PI-88 should prevent islet allograft rejection, disease recurrence in islet isografts (see FIG. 8) and facilitate the survival and function of islet allografts in diabetic NOD/Lt mice. To assess whether PI-88 therapy is graft-protective, NODscid islets were transplanted to autoimmune diabetic NOD mice 400-500 islets/graft. The mice were treated with PI-88 (10 mg/kg/day) i.p. from day 3 post-transplant (after graft revascularisation). Control transplanted mice were treated with saline. Graft function was monitored by measurement of non-fasting blood glucose levels (using a glucometer (MediSense 2)) 2-3×/week. PI-88 treatment of recipient mice prevented recurrence of disease in islet isografts up to 2 weeks post-transplant and permitted these transplants to maintain normoglycaemia; in contrast, control grafts underwent aggressive autoimmune destruction and blood glucose levels in recipient animals returned to the diabetic range by 2 weeks.

Studies conducted by the present inventors have confirmed the presence of a basement membrane (BM) surrounding pancreatic islets in situ, identified perlecan (a heparan sulfate proteoglycan) to be an islet BM component, revealed a 7-fold upregulation in heparanase transcripts in islets from prediabetic and diabetes-onset NOD mice, and found that heparanase inhibition using PI-88 (3) prevents T1D in NOD mice. Thus heparanase produced by activated insulitis MNCs, appears to play a vital role in converting non-destructive insulitis to destructive insulitis by damaging the islet BM and intra-islet ECM, thereby inducing beta cell damage and T1D. Similarly islet isografts are subjected to heparanase-induced immune damage; NODscid islet isografts are protected from disease recurrence in diabetic NOD recipient mice by in vivo treatment with PI-88.

### Example 7

Islet Beta Cell Survival In Vitro is Dependent upon Heparan Sulfate

HSPGs are components of the BM and ECM of pancreatic islets in situ. Earlier Examples have shown that heparanase facilitates the entry of activated MNCs across the islet BM as well as degrades the intra-islet ECM. Such activity appears not only to facilitate the migration of invading MNCs into the islet but, since beta cells appear to be dependent upon ECM heparan sulfate to sustain their viability, reduces the viability of nearby beta cells.

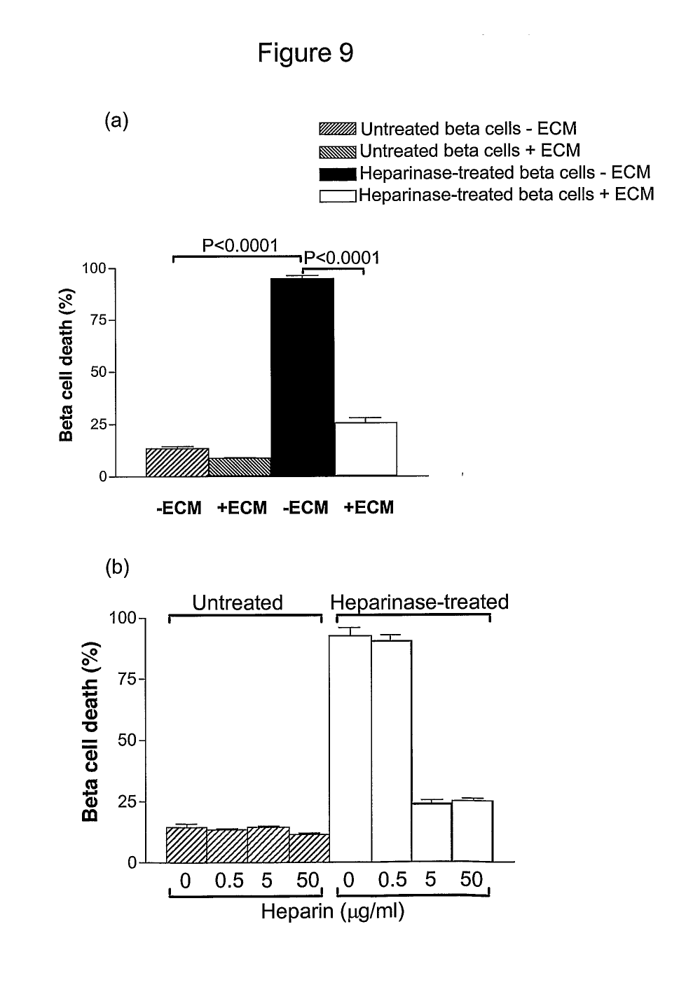

In vitro studies were undertaken to further validate this concept. Isolated BALB/c islets dispersed into single cells using Dispase (1 mg/ml) consist predominantly of insulin-producing beta cells, as confirmed by immunofluorescence. In contrast to control islet beta cells that remained intact over a 2 day culture period, beta cells treated for 1 hr with bacterial heparitinases (heparinases) (I+II+III; with each heparitinase at 0.25 U/ml), a process that would totally destroy HS associated with the islet ECM and cell surface, did not survive (FIG. 9(a)). Placement of treated cells on an ECM (produced in vitro by a cell line) was able to largely rescue the beta cells from heparitinase-induced cell death (P<0.0001) (FIG. 9(a)). These findings indicate that islet beta cells need cell-associated HS to survive. In support of this notion, bacterial heparitinase-treated beta cells were efficiently rescued by providing cultures with 5-50 μg/ml of heparin (*P<0.0001), a highly sulfated form of heparan sulfate (FIG. 9(b)). Islet beta cells therefore require cell-associated HS to remain viable and healthy. These data support the view that, at the cellular level, islet beta cells are susceptible to direct damage by heparanase.

### Example 8

Effect of Heparanase Inhibition with PI-88 on BALB/c Islet Allograft Survival in CBA/H Mice

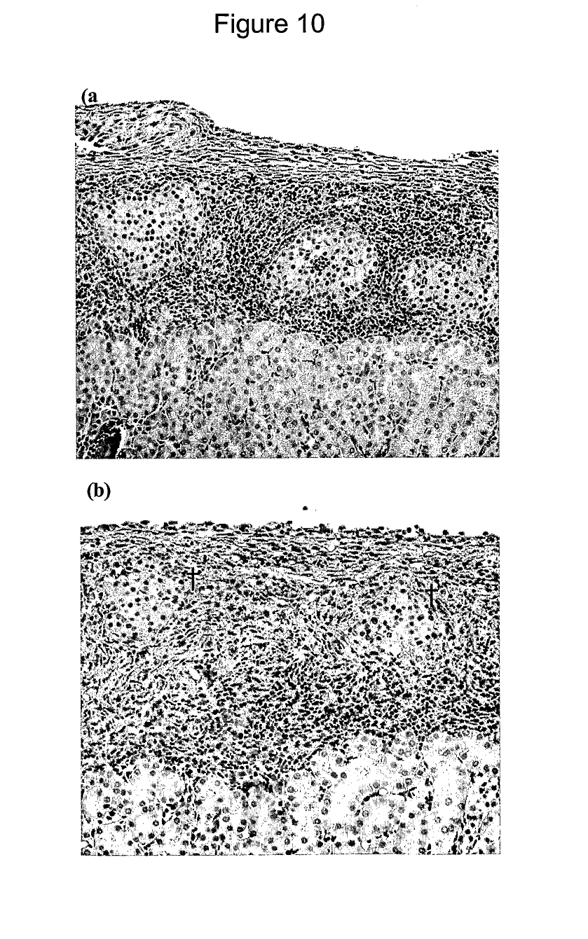

HS plays a critical role in maintaining the integrity and survival of islets and islet beta cells. Heparanase has been shown to play a major role in the autoimmune destruction of islets in NOD mice and exogenous human heparanase can damage normal islets (from conventional mice) in vitro (see earlier Examples). Inhibition of heparanase activity by PI-88 transiently prolongs the aggressive autoimmune destruction of islet isografts in diabetic NOD/Lt mice. It is therefore possible that heparanase also plays an important role in the immunological destruction of islet allografts (even in the absence of autoimmune attack). BALB/c (H-2^{d}) islet allografts from PI-88 treated recipient CBA/H (H-2^{k}) mice at 7 days post-transplant showed accumulation of mononuclear cells around the periphery of islets (FIG. 10(a)), compared to corresponding control islet allografts which showed more advanced islet destruction at 7 days post-transplant (FIG. 10(b)). PI-88-treatment of the host therefore resulted in better preservation of the engrafted allogeneic islets. Heparanase inhibitors therefore represent an anti-rejection strategy for islet transplants in allogeneic hosts and have the capacity to protect grafted islets from both alloimmune and autoimmune attack.

### Example 9

### Compositions for Treatment

In accordance with the best mode of performing the invention provided herein, specific preferred compositions are outlined below. The following are to be construed as merely illustrative examples of compositions and not as a limitation of the scope of the present invention in any way.

### Example 9(A)

### Composition for Parenteral Administration

A composition for parenteral injection could be prepared to contain 0.05 mg to 5 g of a suitable agent or compound as disclosed herein in 10 mls to 2 litres of 1% carboxymethylcellulose.

Similarly, a composition for intravenous infusion may comprise 250 ml of sterile Ringer's solution, and 0.05 mg to 5 g of a suitable agent or compound as disclosed herein.

### Example 9(B)

### Composition for Oral Administration

A composition of a suitable agent or compound in the form of a capsule may be prepared by filling a standard two-piece hard gelatin capsule with 500 mg of the agent or compound, in powdered form, 100 mg of lactose, 35 mg of talc and 10 mg of magnesium stearate.

### REFERENCES

1. Solomon M, Sarvetnick N. (2004) The pathogenesis of diabetes in the NOD mouse. Adv, Immunol. 84: 239.
2. Parish, C R (2006) The role of heparan sulfate in inflammation. Nat Rev Immunol 6: 633.
3. Parish C R, Freeman C, Hulett M D (2001) Heparanase: a key enzyme involved in cell invasion. Biochim. Biophys. Acta 1471: M99.
4. Noonan D M, Fulle A, Valente P et al. (1991) The complete sequence of perlecan, a basement membrane heparan sulfate proteoglycan reveals extensive similarity with laminin A chain, low density lipoprotein-receptor, and the neural cell adhesion molecule. J. Biol. Chem. 266: 22939.
5. Timpi R. (1993) Proteoglycans of basement membranes. Experientia 49: 417.
6. Conde-Knape, K. (2001) Heparan sulfate proteoglycans in experimental models of is diabetes: a role for perlecan in diabetes complications. Diab/Metab Res and Rev 17: 412.
7. Vlodaysky I,. Friedmann Y, Elkin M et al (1999) Mammalian heparanase: gene cloning, expression, and function in tumor progression and metastasis. Nature Med 5: 793.
8. Hulett M D, Freeman C, Hamdorf B J, Baker R T, Harris M J and Parish C R (1999) Cloning of mammalian heparanase, an important enzyme in tumor invasion and metastasis. Nature Med 5: 803.
9. Parish C R. (2005) Heparan sulfate and inflammation. Nature Immunol. 6: 861.
10. Vlodaysky I, Eldor A, Haimovitz-Friedman A et al (1992) Expression of heparanase by platelets and circulating cells of the immune system: possible involvement in diapedesis and extravasation. Invasion metastasis 12: 112.
11. Parish C R, Hindmarsh E J, Bartlett M R et al (1998) Treatment of central nervous system inflammation with inhibitors of basement membrane degradation. Immunol Cell Biol 76: 104.
12. Naparstek Y, Cohen I R, Fuks Z et al. (1984) Activated T lymphocytes produce a matrix-degrading heparan sulfate endoglycosidase. Nature 310: 241.
13. Lafferty K J, Prowse S J, Simeonovic C J et al (1983) Immunobiology of tissue transplantation. A return to the passenger leucocyte concept. Ann. Rev. Immunol. 1: 143.
14. Makhlouf L, Grey S T, Dong V et al (2004) Depleting anti-CD4 monoclonal antbody cures new-onset diabetes, prevents recurrent autoimmune diabetes and delays allograft rejection in nonobese diabetic mice. Transplantation 77: 990.
15. Demirci G, Strom T B, Li X C (2003) Islet allograft rejection in nonobese diabetic mice involves the common gamma-chain and CD28/CD154-dependent and -independent mechanisms. J Immunol 171: 3878.
16. Molano R D, Pileggi A, Berney T et al (2003) Prolonged islet allograft survival in diabetic NOD mice by targeting CD45RB and CD154. Diabetes 52: 957.
17. Wang, Y., Hao, L., Gill, R. G. and Lafferty, K. J. (1987) Autoimmune diabetes in NOD mouse is L3T4 T-lymphocyte dependent. Diabetes 36: 535.
18. Molano R D, Pileggi A, Berney T et al. (2003) Long-term islet allograft survival in nonobese diabetic mice treated with tacrilomus, rapamycin and interleukin-2 antibody. Transplantation 75: 1812.
19. Guo Z, Wu T, Sozen H et al (2003) A substantial level of donor hematopoietic chimerism is required to protect donor-specific islet grafts in diabetic NOD mice. Transplantation 75: 909.
20. Seung, E., Iwakoshi, N., Woda, B. A. (2000). Allogeneic hematopoieic chimerism in mice treated with sublethal myeloablation and anti-CD154 antibody. Blood 95: 217.
21. Lider O, Baharav E, Mekori Y A et al (1989) Suppression of experimental autoimmune diseases and prolongation of allograft survival by treatment of animals with low doses of heparins. J Clin Invest 83: 752.
22. Simeonovic C J, Zarb J C, Gazda L S et al (1998) Pancreatic islet and proislettransplantation in the mouse model. In: Microsurgical methods in rats and mice for transplantation research. Timmermann W, Ulrichs K and Thiede D A (eds). Springer-Verlag, Berlin, Heidelberg, pp 167.
23. Wang R N, Rosenberg L (1999) Maintenance of beta cell function and survival following islet isolation requires re-establishment of the islet-matrix relationship. J Endocrinol 163: 181.
24. Thomas F T, Contreras J L, Bilbao G, Ricordi C et al (1999) Anoikis, extracellular matrix, and apoptosis factors in isolated cell transplantation. Surgery 126: 299.
25. Calvitti M, Baroni, T, Calastrini C et al (2004) Bronchial branching correlates with specific glycosidase activity, extracellular glycosaminoglycan accumulation, TGFbeta2, and IL-1 localization during chick embryo development. J Histochem Cytochem 52: 325.
26. Ihrcke N S, Parker W, Reissner K J et al (1998) Regulation of platelet heparan-ase during inflammation: role of pH and proteinases. J Cell Physiol 175: 255.
27. Freeman C, Parish C R (1998) Human platelet heparanase: purification, characterization and catalytic activity. Biochem J 330:1341.

1-3. (canceled)

4. A method for treating an autoimmune condition in a subject, wherein said method comprises administering a therapeutically effective amount of a heparanase inhibitor to a subject.

5. The method according to claim 4, wherein the autoimmune condition is selected from the group comprising insulitis or diabetes.

6. (canceled)

7. A method of treating or preventing rejection of a transplant in a subject wherein said method comprises administering a therapeutically effective amount of a heparanase inhibitor to a subject.

8. The method according to claim 7, wherein the transplant is a pancreatic islet transplant.

9-11. (canceled)

12. The method according to claim 5, wherein the diabetes is recent-onset type-1 diabetes.

13-18. (canceled)

19. A method for inhibiting the degradation of heparan sulfate in the basement membrane, intra-islet extracellular matrix, peri-islet capsule or any combination thereof in a subject, wherein said method comprises administering a therapeutically effective amount of a heparanase inhibitor to a subject.

20. A method for inhibiting the degradation of heparan sulfate proteoglycan in a subject, wherein said method comprises administering a therapeutically effective amount of a heparanase inhibitor to a subject.

21-24. (canceled)

25. The method according to any of claim 4, 7, 19 or 20, wherein the heparanase inhibitor is selected from the group comprising sulfated polysaccharides, phosphorothioate oligodeoxynucleotides, non-carbohydrate heparin mimetic polymers, sulfated malto-oligosaccharides, phosphosulfomannans, sulfated spaced oligosaccharides, sulfated linked cyclitols, sulfated oligomers of glycamino acids, pseudodisaccharides, siastatin B derivatives, uronic acid-type Gem-diamine 1-N-iminosugars, suramin and suramin analogues, fungal metabolites, diphenyl ether, carbazole, indole and benz-1,3-azole derivatives.

26. The method according to any of claims 4, 7, 19 or 20, wherein the heparanase inhibitor is PI-88.

27. The method according to any of claims 4, 7, 19 or 20, wherein the heparanase inhibitor is a monoclonal antibody.

28. (canceled)

29. (canceled)

30. A composition when used for the treatment or prevention of a condition associated with extracellular matrix degradation, wherein said composition comprises a heparanse inhibitor together with one or more pharmaceutically acceptable carriers, diluents or adjuvants.

31. A composition when used for the treatment or prevention of a condition associated with extracellular matrix degradation, wherein said composition comprises a heparanase inhibitor, together with at least one other immunosuppressant or anti-inflammatory agent and optionally with one or more pharmaceutically acceptable carriers, diluents or adjuvants.

32. The composition of claim 31 wherein said anti-inflammatory agent is selected from the group comprising steroids, corticosteroids, COX-2 inhibitors, non-steroidal anti-inflammatory agents (NSAIDs), aspirin or any combination thereof.

33. The composition of claim 32 wherein said non-steroidal anti-inflammatory agent is selected from the group comprising ibuprofen, naproxen, fenbufen, fenprofen, flurbiprofen, ketoprofen, dexketoprofen, tiaprofenic acid, azapropazone, diclofenac, aceclofenac, diflunasil, etodolac, indometacin, ketorolac, lornoxicam, mefanamic acid, meloxicam, nabumetone, phenylbutazone, piroxicam, rofecoxib, celecoxib, sulindac, tenoxicam, tolfenamic acid or any combination thereof.

34. (canceled)
